(19) United States
(12) Patent Application Publication
(10) Pub. No.: US 2009/0312782 A1
(43) Pub. Date: Dec. 17, 2009

(54) METHOD AND APPARATUS FOR REPAIRING TENDONS

(76) Inventor: MAXWELL CHOONGWON PARK, Calabasas, CA (US)

Correspondence Address: IP GROUP OF DLA PIPER LLP (US), ONE LIBERTY PLACE, 1650 MARKET ST, SUITE 4900, PHILADELPHIA, PA, 19103, US

(21) Appl. No.: 12/138,481

(22) Filed: Jun. 13, 2008

(51) Int. Cl. A61B 17/32

(52) U.S. Cl. 606/184

(57) ABSTRACT

Apparatus for delivering instruments and/or implant structure to a desired location includes a cannula comprising a hollow, elongated body having a first end and a second end, and being sized and adapted to provide access to a targeted location, said first end being eccentrically tapered and defining a targeting extension, the targeting extension having an extension tip that is distal from the cannula body and adapted for temporarily affixing the cannula to the targeted location. The targeting extension is further adapted for direct insertion through soft tissue or tendon, thereby providing access to the targeted location through an opening in the soft tissue or tendon. A novel punch adapted for creating a pilot bole in the targeted location includes a punch tip comprising a pointed extension for piercing and creating an opening in soft tissue or a tendon, thereby providing access to the targeted location.

### FIELD OF THE INVENTION

The present invention relates generally to methods and apparatus for delivering implant structures and for repairing tom tendons. More particularly, the present invention describes methods and apparatus for delivering an implant structure to a targeted location beneath a partially or fully torn tendon, or combination, in a manner that minimizes further trauma to the tendon and facilitates repair of the tendon.

### BACKGROUND

Conventional methods of soft tissue repair (e.g., partial articular-sided tendon tears) are quite time consuming, require a multitude of instruments, and often times cause additional trauma to the very soft tissue being repaired. One known method requires use of a conventional cannula instrument (100), as shown in FIG. 1, in combination with an obturator (200), as shown in FIG. 2 to create a portal of entry through the skin in order to access the surgical site. A suture implant is ultimately anchored and used to suture the damaged soft tissue.

As an initial step in this conventional method, a localizing pin (e.g., a needle) is advanced through the damaged soft tissue to localize and create a path to a targeted location in the bone beneath the soft tissue. The localizing pin is then removed and a punch, and sometimes a tap for relatively hard bone, is advanced through the path created via the localizing needle to the targeted location. As can be appreciated by those in the art, once the localizing pin is removed, the surgeon is left to blindly attempt to advance the punch, and sometimes tap, through the soft tissue along the same path and with the same trajectory as that defined by the localizing pin. Often times, surgeons are unable to duplicate the exact path or trajectory created by the localizing pin resulting in longer surgery times and further trauma to the soft tissue.

Accordingly, it would be desirable to have a method and apparatus for effectively and efficiently repairing soft tissue and/or tendons. It would also be desirable to have a method and apparatus for effectively targeting areas beneath soft tissue or tendons and delivering instruments and/or implant devices thereto, while at the same time minimizing any additional trauma to the soft tissue or tendons.

### SUMMARY OF THE INVENTION

This Application is directed to a method and apparatus for repairing damaged soft tissue or tendons, and in that regard, to a method and apparatus for delivering instruments and/or implant structures to a targeted location beneath the damaged soft tissue or tendon. The novel apparatus for delivering instruments and/or implant structure to a desired location includes a cannula comprising a hollow, elongated body having a first end and a second end. The first end of the cannula may be symmetrically or eccentrically tapered, and it defines a targeting extension. The targeting extension includes an extension tip that is distal from the cannula body and adapted for temporarily affixing the cannula to the targeted location. The targeting extension is also adapted for direct insertion through soft tissue or tendon, thereby providing access to the targeted location through an opening in the soft tissue or tendon.

The apparatus may also include a punch adapted for creating a pilot hole in the targeted location for receiving a suture anchor implant structure. The punch includes a punch tip and is sized and adapted to advance through the cannula body and through the opening in the soft tissue or tendon to the targeted location. The punch tip is itself adapted to penetrate the targeted location and create a pilot hole.

A novel method of delivering an implant structure in accordance with the present invention includes targeting an implant location. Next, a cannula is provided. The cannula preferably comprises a hollow, elongated body having a first end and a second end, and is sized and adapted to provide access to the targeted implant location. The first end of the cannula body may be symmetrically or eccentrically tapered, and defines a targeting extension. The targeting extension includes an extension tip that is distal from the cannula body and adapted for temporarily affixing the cannula to the targeted location. The targeted location is then localized and the targeting extension is temporarily affixed to the targeted location. Then, a punch having a punch tip adapted for penetrating the targeted location and for creating a pilot hole is provided and advanced through the cannula to the targeted location to create the pilot hole. Once the pilot hole is created, the punch is removed from the cannula and an implant structure (or any other instruments/devices) may be delivered through the cannula into the pilot hole created via the punch.

A novel knot-less method of repairing a torn tendon (or any soft tissue) includes delivering one or more suture implant structures to one or more targeted implant locations in a bone area beneath a torn tendon, wherein the delivered implant structures each include one or more suture limbs. Any of the novel cannula and punch described above may be utilized in the delivery. Once delivered, the implant structures are anchored to the targeted areas. Suture limbs emanating from the implant structures are then laterally spanned over the tendon. Each suture limb is then engaged with a limb-anchoring structure, and pulled to tension the suture limbs. The limb-anchoring structures are then introduced and affixed to locations that are lateral and/or distal to the targeted locations, thereby securing and compressing the tendon to its bony attachment site.

### BRIEF DESCRIPTION OF THE DRAWINGS

For the purpose of illustrating the invention, the drawings show forms of the invention that are presently preferred. However, it should be understood that this invention is not limited to the precise arrangements and instrumentalities shown in the drawings.

FIG. 1 shows a conventional cannula;

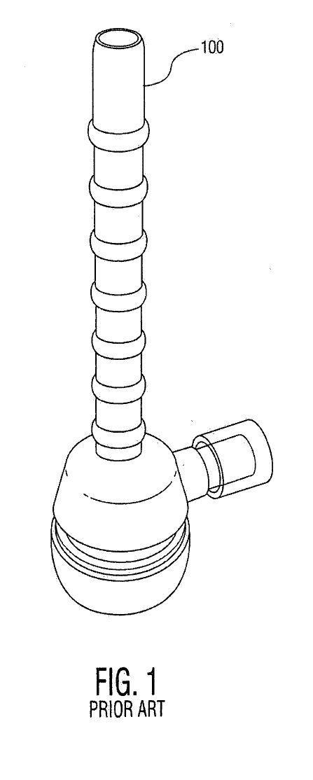

FIG. 2 shows a conventional obturator and;

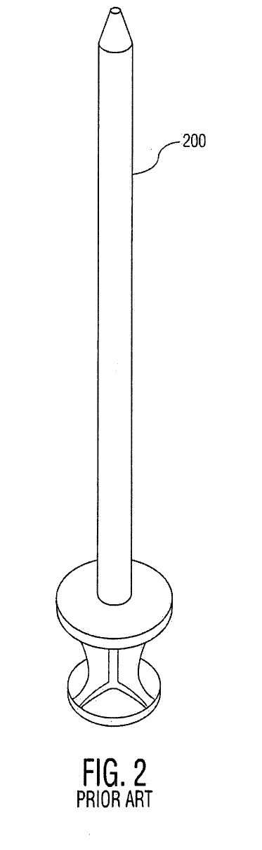

FIG. 3 shows an exemplary cannula instrument configured in accordance with the present invention;

FIG. 4 shows a second exemplary cannula instrument configured in accordance with the present invention;

FIG. 5A shows an exemplary punch apparatus configured in accordance with the present invention;

FIGS. 5B-5C show alternative configurations of the punch apparatus shown in FIG. 5A;

FIG. 6 shows an exemplary cannulated-punch system configured in accordance with the present invention;

FIG. 7 shows a flow diagram representative of an exemplary method of delivering an implant structure in accordance with the present invention;

FIG. 8 shows a diagram that illustrates an exemplary method and apparatus for delivering a screw-type implant in accordance with the present invention;

FIG. 9 shows a diagram that illustrates an exemplary method and apparatus for delivering a toggle implant in accordance with the present invention;

FIG. 10 shows a flow diagram representative of an exemplary method of repairing partially or fully torn soft tissue (or tendon) in accordance with the present invention;

FIG. 11 shows a diagram illustrative of a tendon that has been repaired in accordance with an exemplary method of the present invention using a knotless construct;

FIG. 12 shows a diagram illustrative of a tendon that has been repaired in accordance with an exemplary method of the present invention using an alternate knotless construct; and

FIGS. 13A-13C illustrate an exemplary PASTA lesion repair performed in accordance with the present invention.

### DETAILED DESCRIPTION OF THE INVENTION

Described herein are novel methods and apparatus for use in repairing partially and/or fully torn tendons (e.g., rotator cuff, Achilles, etc.), or a combination thereof (e.g. fully-torn tendon with partial tear component). In general, a tendon repair involves targeting an implant location in a bone area beneath the damaged tendon, creating a pilot hole in the bone, delivering and affixing a suture implant structure into the pilot hole, and then using the implant structure sutures to compress the tendon against the bone. To that end, the present invention describes a novel cannula or tube, a novel punch, and a novel method of utilizing both instruments in a manner that increases surgical efficiency and minimizes soft tissue trauma during a tendon repair procedure.

In a first exemplary embodiment, the present invention relates to a cannula instrument configured for advancing through any soft tissue (e.g., a tendon), for targeting a location in an area beneath the soft tissue (“targeted location”), and for providing a pathway to the targeted location for other instruments useful in repairing the soft tissue (e.g., punch, tap, suture anchor implants, etc.). For purposes of this disclosure, the term “targeted location” simply refers to an area, typically beneath a damaged tendon or soft tissue, that is suitable for advancing and/or anchoring one or more instruments or devices (e.g., suture implant, anchor, suture-locking or interference structure; etc.) in a manner that facilitates repair of the damaged tendon or soft tissue.

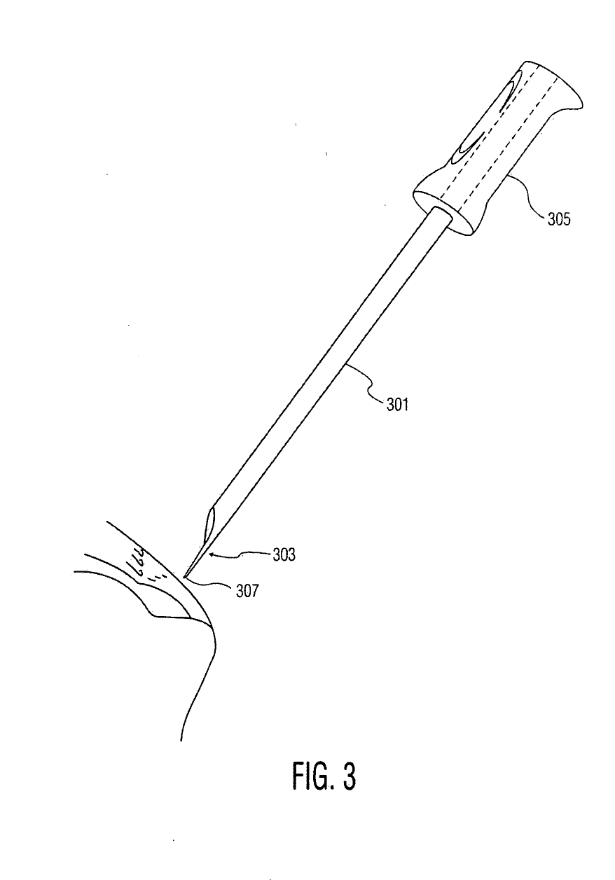

Referring now to FIG. 3, an exemplary cannula (300) configured in accordance with the present invention is shown. The exemplary cannula instrument (300) comprises a rigid, hollowed body (301), a targeting extension (303), and a cannula handle (305). The cannula body (301) may be formed of any material, and in any shape, that is suitable for use during a surgical procedure. For example, the cannula body (301) may be constructed of metal, metal alloy, plastic, polymer, a combination thereof, or any other suitable material, and shaped to define a circular (shown in FIG. 3), elliptical, triangular, rectangular, and/or rhomboidal cross-sectional profile. In one embodiment, the cannula body (301) may be constructed with enough strength and rigidity to withstand being forcibly advanced through soft tissue and/or bone, such as with a mallet or similar device, during a surgical procedure. Additionally or optionally, the interior diameter of the cannula body (301) may be sized and adapted to permit one or more instruments, such as a punch, suture anchor implants, taps, etc., for example, to pass therethrough. Suture anchor implants, as the term is used herein, refers generally to any type of implant structure (whether known or unknown) that is usable for attaching soft-tissue to bone. Screw-type, plug-type, barb-type, expandable-type, and toggle-type implants, for example, are known types of implant structures. For the purposes of this disclosure, it is to be understood that the terms “implants” and “structures” refer to any implant, structure, and/or device that provides, affixes, and/or delivers sutures to bone and soft tissue. It is anticipated that future and/or “to-be developed” types of implant structures (and other instruments) may also be used in conjunction with the exemplary cannula (300) instrument of the present invention. Also, it is to be understood that the term “sutures”, as used herein, refers to any form of sutures including, but not limited to, cord-type and band-type (i.e., flat, tape-like) suture configurations, both of which may consist of braided or woven aspects or components, in combination, without limitation.

At one end, the cannula (300) may be eccentrically tapered so as to define a targeting extension (303) adapted for directed insertion through soft tissue (e.g., tendon) to a targeted location beneath the soft tissue. The exemplary targeting extension (303) further defines an extension tip (307) that is distal from the cannula body (301) and is adapted for temporarily affixing the cannula (300) to the targeted location. Since the targeted location is often a hard, bony surface, the extension tip (307) may also be constructed of a strong, durable and rigid material.

As shown in FIG. 3, the targeting extension (303) of the exemplary cannula (300) may be configured as a tapered extension having a contour consistent with the curvature of the cannula body (301). It should be understood, however, that the targeting extension (303) may be configured in any shape, length, and/or contour as appropriate for the particular implementation. As an example, the targeting extension (303) may be configured as a long, narrow pin or needle adapted to pierce soft tissue, minimizing soft tissue injury, and to localize a targeted location beneath the soft tissue, all without causing any additional trauma to the soft tissue itself. Alternatively, the targeting extension (303) may be configured in any number of desired forms. To illustrate, the tip (307) may be configured to form a “tooth”-type tip, a thin and flat tip, a tapered tip a beveled tip, or any other tip (307) configuration that facilitates penetrating soft tissue with minimal trauma, parallel to its fibers in certain cases or that is suitable for the particular application.

In an alternate embodiment, one or both of the targeting extension (303) and/or the extension tip (307) may be configured as one or more independent components that may be added to existing cannula instruments. In such an embodiment, the targeting extension (303) and/or its tip (307) may be mounted, clipped, clamped, or otherwise fastened to existing cannula instruments in order to achieve the effect of the cannula instrument of the present invention. With regard to the extension tip (307), the tip (307) may be formed as a long and narrow needle-like instrument that may be inserted or removed from the cannula (300) through an opening that extends the entire length of cannula (300). In such an embodiment, the cannula (300) may be formed to define an additional opening that begins at one end and that extends along the entire length of the cannula (300). This opening may be sized to allow a needle-like extension tip (307) to enter the cannula (300) at one end and protrude through to the targeting extension (303) area.

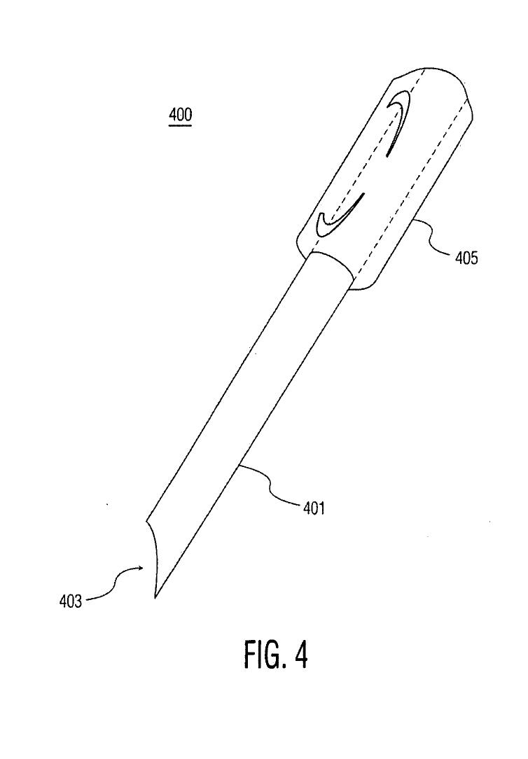

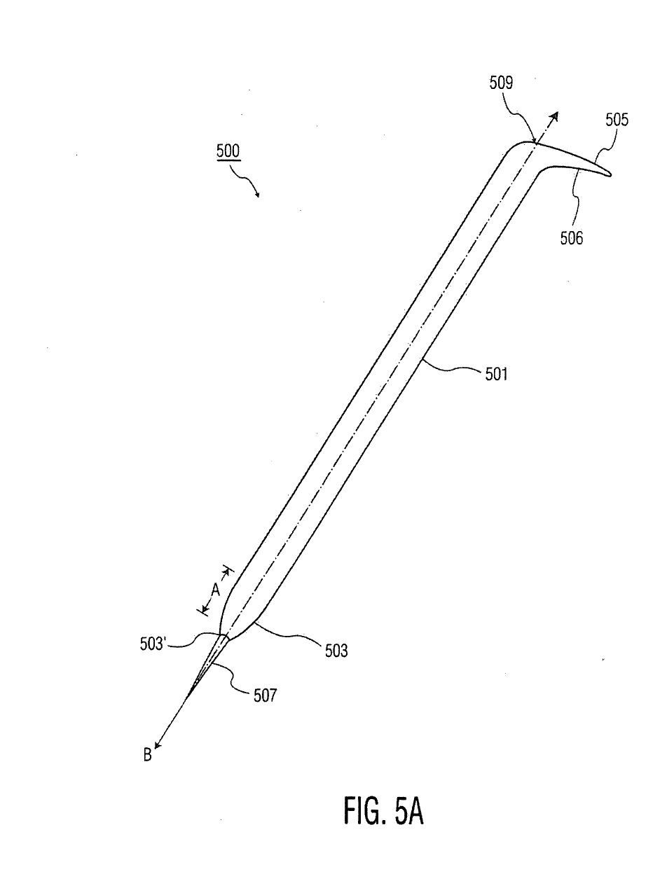

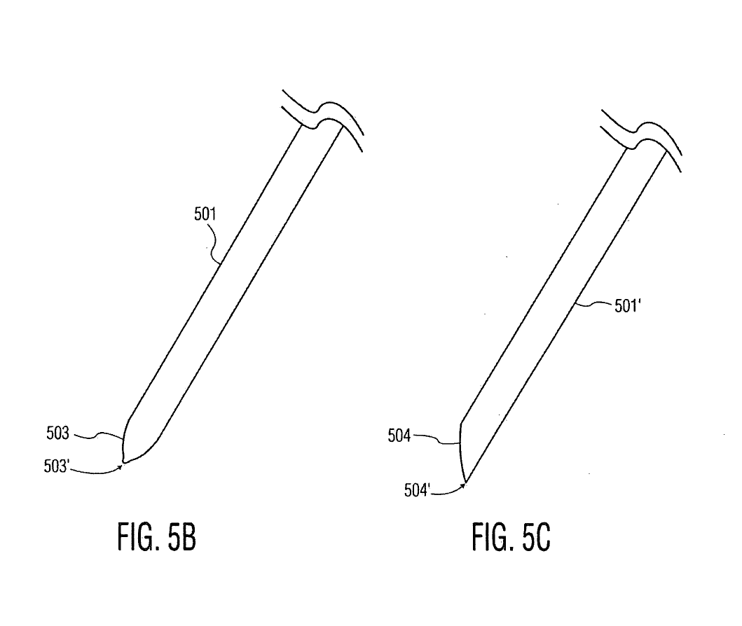

Turning briefly to FIG. 4, an exemplary cannula (400) configured with a tooth-type extension (403) is shown. Similar to the previously described cannula (300), the instant cannula (400) comprises a rigid, hollowed body (401), a targeting extension (403), and an optional cannula handle (405). Rather than a narrowed extension (303), however, the cannula (400) of FIG. 4 comprises a tooth-type extension or beveled extension (403). As can be appreciated by those in the art, certain implementations may be better suited for utilizing this tooth-type extension (403) than others. Implementations requiring forcible advancement of the cannula (400), for example, may be better suited to include a tooth-type tip (403) rather than a long, narrow pin-type tip. Further, for implementations in which a separate pin-instrument is utilized to navigate through soft tissue (e.g., see novel punch of FIG. 5A), a cannula having a long extension tip may not be necessary. Instead, a tooth-type or beveled cannula (400) may be preferred.

Returning again to FIG. 3, the exemplary cannula (300) also comprises a cannula handle (305) attached to the cannula body (301) at an end opposite that of the targeting extension (303). The optional handle (305) may be constructed as part of the cannula itself, or it may be constructed separately and then attached to the cannula body (301). Similar to the cannula body (301), the optional handle (305) may be constructed of stainless steel, metal, metal alloy, plastic, polymer or any other material that is suitable for use in surgical procedures. Optionally, as is shown in FIGS. 3 and 4, the cannula handle (305, 405) may be asymmetrically formed for tactile orientation with respect to the targeting extension (303, 403), thus facilitating visual-tactile feedback to the operating surgeon. The handle (305) may also be formed with one or more contours or depressions to facilitate holding or gripping the cannula (300) in place.

Utilizing any of the novel cannula instruments described above (see FIGS. 3 or 4) provides significant advantages over existing instruments, particularly with respect to soft tissue and/or tendon repair procedures. As previously explained, conventional methods of soft tissue repair (e.g., partial articular-sided tendon tears) require that a localizing pin, followed by a punch instrument be advance through the soft tissue to localize and create a path (i.e., pilot hole) to a targeted location beneath the soft tissue. Once the localizing pin and punch are removed, the surgeon is left to blindly attempt to advance additional instruments (e.g., suture implants, taps, etc.) through the soft tissue along the same path and with the same trajectory as that defined by the pilot hole. Often times, since surgeons are unable to see the necessary instruments until they pass through the soft tissue or tendon, they are required to blindly navigate for the proper path/trajectory to the target location or the pilot hole. As can be appreciated by those in the art, this blind navigation typically results in longer surgery times and further potential trauma to the soft tissue.

The novel cannula of this invention, in sharp contrast, provides a targeting extension that enables surgeons to locate and target areas beneath soft tissue while ensuring that additional instruments advanced through the soft tissue follow the path created by the targeted extension, thereby increasing the surgeons' efficiency and minimizing soft tissue trauma. To illustrate, reference is again made to FIG. 3. In a procedure to repair a partial articular-sided tendon avulsion (PASTA) lesion, for example, the targeting extension (303) may be advanced through the damaged tendon to locate an area beneath the tendon that is suitable for anchoring one or more suture anchor implants. For purposes of this illustration, any type of suture anchor implant may be utilized in connection with the exemplary cannula (300) including, without limitation, screw-type implants, plug-type implants, toggle-type implants, suture-locking or -interference structures, etc. As will be understood by those in the art, suture limbs emanating from these anchor implants may then be utilized to secure and compress the damaged tendon to its attachment site.

Once the targeted extension (303) is advanced through the tendon, rather than removing it from the tendon, the targeted extension (303) may be temporarily affixed to the targeted area, thereby providing a fixed pathway through which the suture anchor implants (and/or any other instruments) may advance. Since the cannula (300) does not have to be removed from the tendon, the surgeon does not have to blindly navigate for a pathway or pilot hole. Instead, the surgeon is able to advance instruments such as a punch, tap, suture anchors, etc. through the body (301) of the cannula (300) along the exact same path and trajectory as that created by the targeted extension (303). As can be appreciated by those in the art, utilizing a cannula (300) configured in this manner provides a visual-tactile feedback to the surgeon for implant placement. Providing this visual-tactile feedback enables the surgeon to limit the number of times that instruments are passed through the tendon, resulting in reduced trauma to the tendon and in a more efficient surgical procedure.

Referring now to FIG. 5A, an exemplary punch instrument (500) configured in accordance with the present invention is shown. This exemplary punch (500) may be utilized, for example, as part of a surgical procedure directed at repairing soft tissue injuries (e.g., rotator cuff injuries). To that end, the exemplary punch (500) is configured for advancing through soft tissue and for penetrating, and creating an opening (or pilot hole) in a hard surface (e.g., a bony targeted location) beneath the soft tissue. This opening may then be utilized for receiving and anchoring a surgical device, such as a suture implant or suture-locking or -interference structure, for use in repairing the soft tissue injury. As further discussed below, the exemplary punch (500) may be used alone, or in combination with one of the novel cannula instruments discussed above as part of a cannula-punch system.

The exemplary punch instrument (500) comprises a shaft (501), a punch tip (503) at one end of the shaft (5.01), and an optional punch lid (505) at the other end of the shaft (501). Also included in this exemplary punch instrument (500) is an optional punch extension tip (507). The punch shaft (501) may be formed of any material and formed in any shape that is suitable for use during a surgical procedure. For example, the punch shaft (501) may be constructed of stainless steel, metal, metal alloy, plastic, polymer, a combination thereof, or any other suitable material, and shaped to define a circular (shown in FIG. 5), elliptical, triangular, rectangular, and/or rhomboidal cross-sectional profile. In addition, exemplary punch shaft (501) may be constructed with enough strength and rigidity to withstand being forcibly advanced through soft tissue and/or bone, such as with a mallet or similar device, during a surgical procedure. Additionally or optionally, the diameter of the punch shaft (501) may be sized according to the particular implementation. For example, a smaller-diameter punch (500) may be preferred for surgeries involving children, while a larger diameter punch (500) may be used for adults. The size of the particular implant to be utilized will dictate the preferred diameter of the punch.

At one end of the punch shaft (501) is a punch tip (503). The punch tip (503) is configured for penetrating and/or advancing through soft tissue (e.g., tendon), and for penetrating and creating an opening (e.g., a pilot hole) in a hard surface (e.g., a bony targeted location) beneath the soft tissue. As noted above, the opening (e.g., in a bone area, through soft tissue) created by the punch tip (503) may be utilized for receiving and anchoring a surgical device such as a suture implant, or suture-locking or -interference structure. As shown in FIGS. 5A and 5B, the punch tip (503) may be symmetrically tapered (taper A) such that its point (503′) converges along the punch's (500) center axis B. Alternatively, as shown in FIG. 5C, the punch tip (504) may be eccentrically tapered, such that the point (504′) of the punch tip (504) favors and approaches the punch's (500) outer perimeter. In such an embodiment, the punch tip (504) may be configured in various forms, to include but not limited to, a relatively thin and flat tip, a beveled tip, or tapered tip that facilitates penetrating soft tissue with minimal trauma. For punch configurations that utilize an eccentrically placed tip (as in FIG. 5C), the punch (500) may optionally comprise a punch handle (not shown) that is asymmetrically formed for tactile orientation with respect to the distal tip (504′), thus facilitating visual-tactile feedback to the operating surgeon. The handle may also be formed with one or more contours or depressions to facilitate holding or gripping the punch in place.

Offsetting the punch point (504′) as illustrated in FIG. 5C may be useful in decreasing the amount of trauma inflicted on soft tissue as the punch itself is advanced through the soft tissue. To illustrate, suppose an eccentrically tapered punch (see 5C) were used in conjunction with the cannula instrument (300) of FIG. 3 during a surgical procedure for repairing a PASTA lesion. The cannula instrument (300) would be used to create an initial opening in the tendon through which the targeting extension (303) may be advanced in order to locate a bony area beneath the tendon. The punch could then be advanced through the cannula (300) with its eccentrically tapered punch tip (504′) adjacent to the cannula targeting extension (303). As can be appreciated by those skilled in the art, orienting the punch tip (504′) in this manner eliminates the need for the punch to create a second opening in the tendon for advancing. Instead, the punch could enter the tendon through the same opening created by the cannula targeting extension (303). As a result, the tendon would experience less trauma.

Returning now to FIG. 5A, the exemplary punch instrument (500) includes an optional punch extension tip (507). This extension tip (507) may be formed as part of the punch (500) itself, or it may be an independent component that is attached (and removed) from the punch tip (503). Alternatively, the extension tip (507) may be formed as a long and narrow needle-like instrument that may be inserted or removed from the punch (500) through an opening that extends the entire length of punch (500). In such an embodiment, the punch (500) may be formed to define an opening that begins at one end (509) and that extends along the entire length of the punch (500) through the punch center axis (B). This opening may be sized to allow a needle-like extension tip (507) to enter the punch (500) through its top (509), advance through the punch shaft (501), and protrude through the punch tip (503) area. In this type of embodiment, the needle-like punch tip (507) may be utilized to initially penetrate damaged soft tissue in order to localize a targeted area beneath the soft tissue (such as in a PASTA lesion surgery). Once the targeted area is located, the needle-like punch tip (507) may be removed (through the punch shaft (501)) and the punch (500) may be advanced through the opening created by the needle-like tip (507).

Also included in the exemplary punch instrument (500) is an optional punch “lid” (505). The punch lid (505) may be formed of any durable, rigid material suitable for surgery and for receiving blunt force or pressure for forcibly advancing the punch (500), such as with a mallet or other instrument. In one embodiment, the punch lid (505) may be configured for receiving reverse force (e.g., back taps) for reversing the punch's (500) direction of advancement. To illustrate, if the punch (500) were used to penetrate and create an opening in a bone, one could “back-tap” the underside (506) of the punch lid (505) to facilitate removal of the punch (500) from the bone. In such an embodiment, the punch lid (505) may be sized and strengthened enough to receive reverse force, such as from a mallet or other instrument. Although the exemplary punch lid (505) is shown extending to one side of the punch (500), it should be understood that the punch lid may be configured to extend in any direction relative to the punch (500), or not at all, and may be configured as part of a handle.

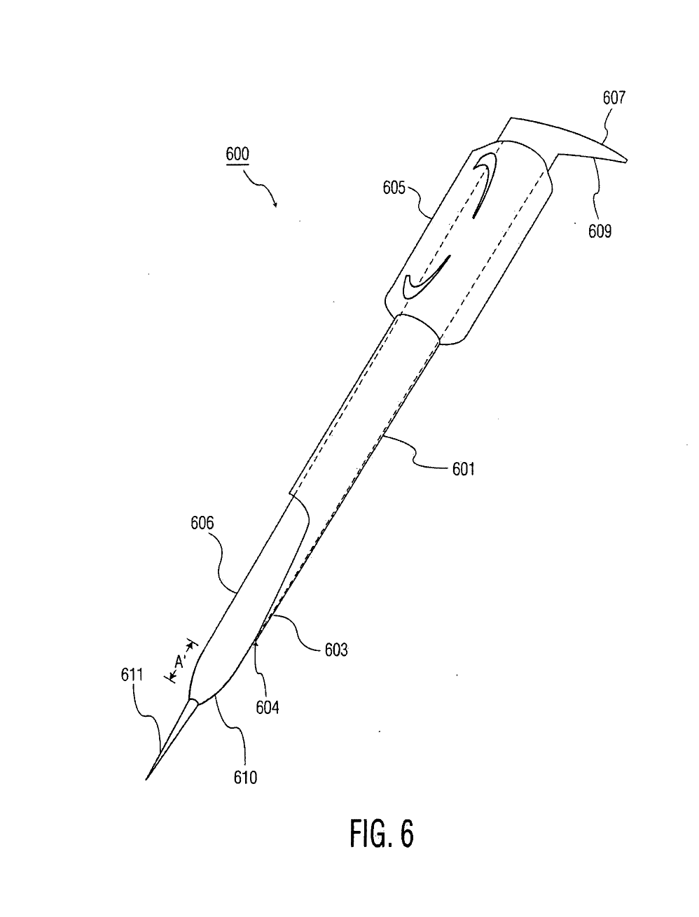

As noted above, each of the novel cannula instruments and punch instruments described above may be utilized independently, or in combination with one another. In one embodiment, the exemplary cannula (300) of FIG. 3 may be combined with the exemplary punch instrument (500) of FIG. 5A to form a cannula-punch system (600), as is shown in FIG. 6. In another embodiment, the exemplary cannula (400) of FIG. 4 may be combined with the exemplary punch instrument (500) of FIG. 5A to form an alternate cannula-punch system. For purposes of illustration, the cannula-punch system (600) shown in FIG. 6 will be discussed in detail.

Turning now to FIG. 6, an exemplary cannula-punch system (600) is shown. The cannula-punch system (600) includes a cannula portion primarily comprising a hollowed cannula body (601), a targeting extension (603), and an optional cannula handle (605). At one end, the cannula portion is eccentrically tapered so as to define a targeting extension (603) adapted for directed insertion through soft tissue (e.g., tendon) to a targeted location beneath the soft tissue. The targeting extension (603) further defines an extension tip (604) that is distal from the cannula body (601) and is adapted for temporarily affixing the cannula portion of the cannula-punch system (600) to the targeted location. It should be noted that although the exemplary targeting extension (603) is shown as a long, narrow extension, the targeting extension (603) may be configured in any shape, length, and/or contour as appropriate for the particular implementation. As an example, the targeting extension (603) may be configured as a “tooth” type extension, such as is shown and discussed above in FIG. 4. Implementations requiring forcible advancement of the cannula (400), for example, may be better suited to include a tooth-type tip (403) rather than a long, narrow pin type tip.

Returning to FIG. 6, the cannula portion of the exemplary cannula-punch system (600) also comprises an optional cannula handle (605) attached to the cannula body (601) at an end opposite that of the targeting extension (603). The optional handle (605) may be constructed as part of the cannula itself, or it may be constructed separately and then attached to the cannula body (601). Similar to the cannula body (601), the optional handle (605) may be constructed of stainless steel, metal, metal alloy, plastic, polymer or any other material that is suitable for use in surgical procedures. Optionally, the cannula handle (605) may be asymmetrically formed for tactile orientation with respect to the targeting extension (603). The handle (605) may also be formed with one or more contours or depressions to facilitate holding or gripping the cannula-punch system (600) in place.

Inserted through the center of the cannula body (601) is the punch portion of the cannula-punch system (600). The punch portion primarily comprises a shaft (606), a punch tip (610) at one end of the shaft (606), and an optional punch lid (607) at the other end of the shaft (606). Also included in the exemplary punch is an optional punch extension (611).

The punch tip (610) is configured for penetrating and/or advancing through soft tissue (e.g., tendon), and for penetrating and creating an opening (e.g., a pilot hole) in a hard surface (e.g., a bony targeted location) beneath the soft tissue. In the present illustration, the punch tip (610) is shown to be symmetrically tapered (taper A′) such that its point converges along the punch's center axis. Alternatively, the punch tip (610) may be eccentrically tapered along the same angle as that of the cannula targeting extension (603) and tip (604)

J At one end of the punch portion, attached to the end of the punch tip (610), is an optional punch extension (611). The punch extension (611) may be formed as part of the punch tip (610) itself, or it may be an independent component that is attached (and removable) from the punch tip (610). Alternatively, the extension (611) may be formed as a long and narrow needle-like instrument that may be inserted or removed from the punch through an opening that extends the entire length of the punch itself. In such an embodiment, the needle-type punch extension (611) may be utilized to initially penetrate a damaged soft tissue in order to localize a targeted area beneath the soft tissue (such in a PASTA lesion surgery). Once the targeted area is located, the needle-like punch extension (611) may be removed (through the punch shaft (606)) and the punch may be advanced through the opening created by the needle-like extension (611). The needle may be removed after the punch is advanced as well.

Attached to the other end of the punch portion is an optional punch lid (607). The punch lid (607) may be formed of any durable, rigid material suitable for surgery and for receiving blunt force or pressure for forcibly advancing the punch into a hard surface (e.g., bone), such as with a mallet or other instrument. As shown, the punch lid (607) is configured for receiving reverse force (e.g., back taps) on an underside (609) of the punch lid (607) for reversing the punch's direction of advancement. The punch lid (607) is sized and strengthened enough to receive reverse force, such as from a mallet or other instrument. Although the exemplary punch lid (607) is shown extending to one side of the cannula-punch system (600), it should be understood that the punch lid (607) may be configured to extend in any direction relative to the cannula-punch system (600), or not at all, and may be configured as part of a handle.

All components of the cannula-punch system (600) may be formed of any material, and in any shape, that is suitable for use during a surgical procedure. For example, the punch and cannula portions (or any components comprising the same) of the system (600) may be constructed of stainless steel, metal, alloy, plastic, polymer, a combination thereof, or any other suitable material, and shaped to define a circular (shown in FIG. 5), elliptical, triangular, rectangular, and/or rhomboidal cross-sectional profile. In addition, the cannula-punch system (600) may be constructed with enough strength and rigidity to withstand being forcibly advanced (and/or removed) through soft tissue and/or bone, such as with a mallet or similar device, during a surgical procedure.

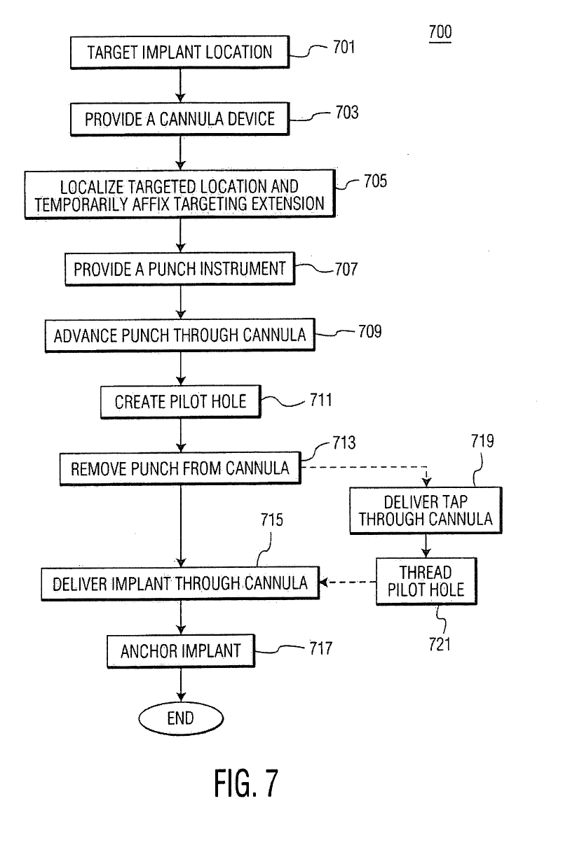

In operation, the exemplary cannula-punch system (600) may be utilized as part of a soft tissue (partial or full tear, or combination (e.g. full-thickness tear with a partial-tear component)) repair surgery, for example, to advance and deliver one or more instruments to a targeted area beneath the damaged soft tissue. In one embodiment, the cannula-punch system (600) may be used to deliver one or more suture anchors or implants, or suture-interference or—locking structures to a targeted location in accordance with the novel delivery method described below and illustrated in FIG. 7.

Turning now to FIG. 7, an exemplary flow diagram representative of a novel method (700) of delivering an implant structure is shown. According to the method (700), a first step in delivering an implant structure (for use in repairing a damaged soft-tissue or tendon) includes targeting an implant location (step 701) beneath the damaged soft-tissue or tendon (e.g., rotator cuff tendon). In the case of a partially-torn tendon repair surgery (e.g., a PASTA lesion surgery), for example, targeting an implant location (step 701) may include piercing and creating an opening in the soft tissue or tendon, such as with a needle or similar device. Alternatively, in the case of a fully-torn tendon repair surgery, this step (step 701) may simply comprise visually identifying and targeting the implant location. However, for fully-torn tendon repair surgery, piercing and creating an opening in the soft tissue or tendon is not precluded, and is in certain cases preferred.

J Next, in step 703, a cannula device is provided. This cannula device preferably comprises a hollow, elongated body having a first end and a second end, and being sized and adapted to provide access to the targeted implant location. The first end of the cannula device may be eccentrically tapered defining a targeting extension that is distal from the cannula body and adapted for temporarily affixing the cannula to the targeted location (e.g., see FIG. 3). Alternatively, the cannula device may comprise a tooth-type targeting extension (e.g., see FIG. 4). The cannula device may also comprise an optional handle, located a the second end of the cannula body for providing tactile orientation with respect to the targeting extension.

Once the cannula device is provided (step 703), the targeted location is localized and the targeting extension is advanced through the soft tissue or tendon (if necessary) and is temporarily affixed to the targeted location (step 705). This localizing and temporarily affixing step may be accomplished, for example, by piercing and creating an opening in the soft tissue or tendon, and by inserting the extension tip through the opening created in the soft tissue or tendon, respectively. As an option, a blunt-tip obturator may be provided and used to palpate the soft tissue or tendon just prior to piercing-and/or advancing the extension tip through the soft tissue or tendon. This will limit potential trauma to the soft tissue or tendon resulting from the extension tip, as the blunt obturator will help localize the area where the extension is to be advanced by providing visual-tactile feedback to the surgeon.

In one embodiment, the initial opening in the soft tissue or tendon may be created using a localizing needle, for example. Alternatively, the targeting extension may be configured with a pointed extension tip, in which case the initial opening may be created using the cannula targeting extension. In such an embodiment, the targeting (step 701) and localizing (step 705) steps may occur simultaneously using the pointed extension tip.

Next, a punch instrument is provided (step 707) and advanced through the cannula device step 709) to the targeted location. The punch instrument preferably includes a punch tip at one end that is adapted for penetrating soft tissues (e.g., tendon) and bard surfaces (e.g., bone), and for creating a pilot hole therein. Such a punch tip may be symmetrically tapered such that the punch tip converges along the punch's central axis, or the punch tip may be eccentrically tapered to follow the same line or projection as that of the cannula device targeting extension.

Optionally, the punch instrument may be further configured to include a needle-like punch extension that extends laterally in the same direction as the cannula targeting extension. This needle-like punch extension may be formed as part of the punch tip itself, or it may comprise an independent component that may be attached and/or removed from the punch tip. Alternatively, the punch extension may be formed as an independent needle-like instrument that may be inserted and removed through an opening that extends the entire length of the punch instrument itself. Punch instruments having this type of needle-like punch extension may be utilized to simultaneously target and localize the implant location (steps 701 and 705, respectively).

Once the punch is advanced through the cannula (step 709), the punch may be forcibly advanced into the targeted location (step 711) using a mallet or similar instrument, thereby creating a pilot hole. Notably, if the punch is advanced through soft tissue or tendon, then a pilot hole is also created in the soft tissue (in addition to bone). Next, the punch instrument may be removed from the cannula (step 713). To facilitate removal of the punch instrument (step 713), the punch instrument may further comprise an optional punch lid positioned at an end opposite that of the punch tip. This punch lid may include an undersurface configured for receiving reverse force (e.g., back tapping force from a mallet or similar instrument) for extracting the instrument if it becomes lodged in a bony targeted surface.

Upon removing the punch instrument from the cannula (step 713), an implant structure may be delivered (step 715) through the cannula device to the pilot hole created in the targeted location. Once delivered (step 715), the implant structure may be anchored (step 717) according to any known method of anchoring such a device. For implementations desiring to deliver screw-type implant structures (or any implant requiring the use of a tap prior to placement), the method (700) may further comprise (prior to delivering the implant structure (step 715)) the steps of: providing a tap instrument and delivering the tap instrument to the targeted location through the cannula device (step 719); and “threading” the pilot hole to receive a screw-type, or other appropriate, implant (step 721).

In an alternate implementation, the cannula device and punch instrument may be replaced with a cannula-punch system (e.g., see FIG. 6). In such an implementation, the cannula-punch system may be utilized to simultaneously target and localize the implant location (steps 701 and 705, respectively). In addition, by utilizing a cannula-punch system, the need to provide a cannula device (step 703), then separately provide a punch instrument (step 707) would be eliminated. Instead, both devices (i.e., the cannula and punch) could be provided in a single step, thereby reducing the number of steps required to accomplish the delivery method (700).

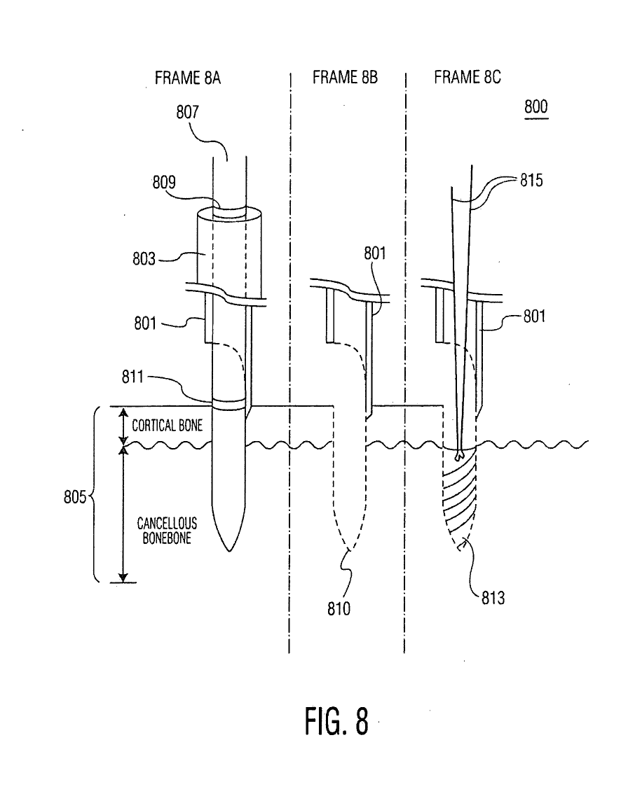

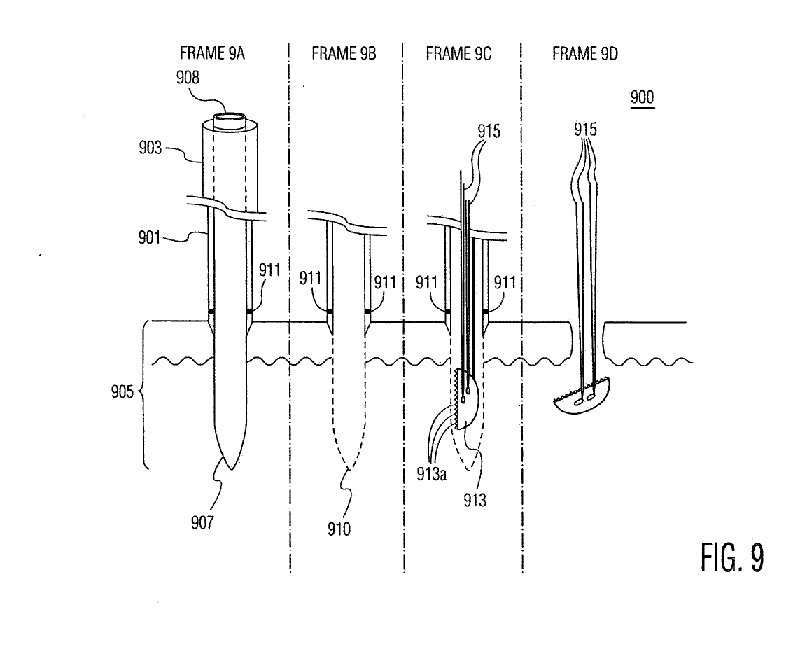

Referring now to FIGS. 8 and 9, two diagrams representing illustrative examples of embodiments of the present invention are shown. The diagram (800) of FIG. 8 illustrates an exemplary method and apparatus for delivering a screw-type implant in accordance with the present invention; and FIG. 9 shows a diagram (900) depicting an exemplary method and apparatus for delivering a toggle-type implant. Both FIGS. 8 and 9, without limitation, may be applied to PASTA lesions, full-thickness tendon tears, and combinations (e.g., full-thickness tear with a partial tear component).

Looking first to FIG. 8, the diagram (800) depicts the delivery of a screw-type implant (813) using methods and apparatus of the present invention in three sequential frames: 8A, 8B, and 8C In frame 8A, a cannula instrument (801) is shown partially advanced through a bony surface (805). The cannula instrument (801) comprises a long, tapered targeting extension whose circumference is less than that of the cannula body and a handle (803) for use in managing or advancing the cannula (801).

As depicted in frame 8A, only the targeting extension has been advanced into the bony surface (805). In this manner, the cannula (80,1) may be temporarily affixed to the bony surface (805) without having to advance the full diameter of the cannula (801) into the bone (805). As can be appreciated by those in the art, advancing the full diameter of the cannula (80 1) into the bone may create a hole larger than threads of the screw-type implant, thereby compromising the fixation of the suture anchor implant. Once the cannula (801) is properly positioned, a punch (807) is shown advanced through the cannula (801) into the bony surface (805), thereby creating a pilot hole (810) for receiving the screw-type implant (813). Laser lines (809, 811) on the punch provide guidance for advancing the punch (807) to the correct depth.

Once the pilot hole (810) is created, the punch is removed through the cannula (801), as depicted in frame 8B. It is noted, however, that the cannula (801) remains temporarily affixed or engaged to the bony surface (805). By maintaining the cannula (801) temporarily affixed in place, the cannula (801) is able to provide a single guided pathway to the pilot hole (810) for delivering the screw-type implant (813).

Moving now to frame 8C, once the punch (807) has been removed, a tap device may be used to thread the pilot hole (not shown). The screw-type implant (813) may then be delivered through the cannula (801) and anchored in place. Once the implant (813) is anchored, the cannula (801) may be removed, and the implant sutures. (e.g., cord-like or band-like sutures) (815) may be utilized to affix soft tissue (not shown) to the bony surface (805).

Referring now to FIG. 9, an illustrative diagram (900) depicting an exemplary method and apparatus for delivering a toggle-type implant in accordance with the present invention is shown. According to the diagram (900), the delivery of a toggle-type implant (913) may be depicted in four sequential frames: 9A, 9B, 9C, and 9D. In frame 9A, a cannula instrument (901) is shown partially advanced into a bony surface (905) (e.g., through cortical bone); the portion of the cannula depicted meeting or engaging the bone (905) represents the cannula extension and tip. The cannula instrument (901) comprises a tooth-like or beveled targeting extension (not shown) whose circumference is less than that of the cannula body, and a handle (903) for use in managing and advancing the cannula (901). Notably, a targeting extension may be used on the punch (not shown).

As depicted in frame 9A, a punch obturator (907) is used in conjunction with the cannula (901) to simultaneously advance the cannula (901) and the punch obturator (907) into the bony surface (905). A ridge (908) on the top of the punch obturator (907) is used to drive the cannula (via the cannula handle (903)) into the bone (905) as the punch obturator (907) is itself struck with a mallet or similar instrument. Advancing the cannula (901) into the bone (905) in this manner temporarily affixes the cannula (901) in the bone (905). Laser lines (911) on the exterior of the cannula (901) may be used for guidance when advancing the cannula (901) to a correct depth. Alternatively, the cannula (901) need not be forcibly advanced, but rather engaged to the bone (905) via an extension and tip, in which case the ridge (908) would not be necessary and forcible advancement would be provided solely to the punch (907) component instrument directly.

Once the cannula (901) is properly advanced and affixed in the bone (905), the punch obturator (907) is shown removed from the cannula (901), thereby exposing a pilot hole (910) created in the bone (905) for receiving the toggle-type implant (913) (frame 9B). The toggle implant (913) is then ready to be advanced through the cannula (901) into the pilot hole (910), as depicted in frame 9B.

As shown, but not limited to this depiction, the toggle-type implant (913) includes at least two sutures (915) attached to each of two or more corresponding eccentric eyelets (914) for use in toggling the implant (913) once it is properly positioned. The toggle-type implant (913) also includes one or more barbs (913a) for use in anchoring the implant (913) to an undersurface of the bone (905) once the implant (913) is toggled or deployed.

Next, as depicted in frame 9C, toggle-type implant (913) is inserted and advanced along the cannula into the pilot hole (910). As depicted in frame 9D, the implant (913) is then toggled and the cannula (901) is fully removed from the bone (905). Once the implant (913) has been toggled, the sutures (915) are pulled to firmly engage the anchor implant barbs (913a) to the underside of the bone (905), thereby firmly anchoring the implant (913) in position. Once properly positioned, the implant sutures (e.g., cord-like or band-like) (915) may be utilized to securely attach soft tissue (not shown) to the bony surface (905).

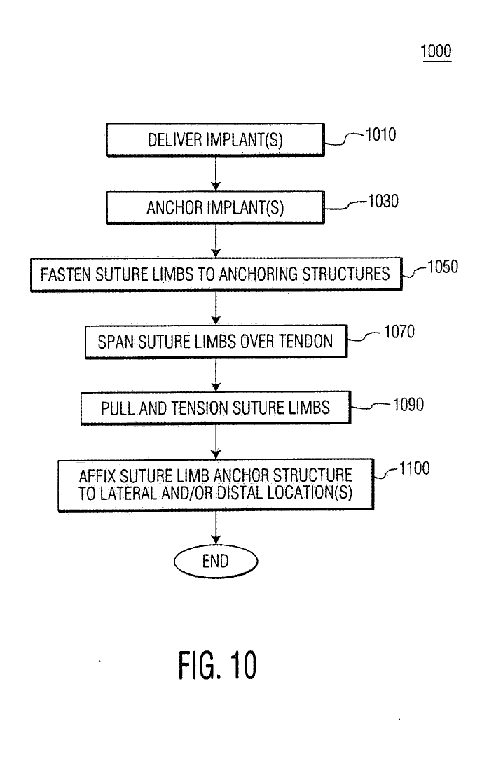

Referring now to FIG. 10, an exemplary flow diagram illustrating a novel method (1000) of repairing a partially or fully torn tendon (e.g., rotator cuff) is shown. Unlike conventional tendon repair methods, which utilize some form of knotting technique for securing torn tendons to bone, the method (1000) of this invention enables surgeons to repair tendons without using any knots. As a result, the method (1000) of this invention reduces the surgical time necessary for performing such procedures, and it improves tendon-bone contact and fixation. Although this method (1000) will be described in the context of a partially-torn tendon repair, it should be understood that the present method (1000) may be utilized in repairing full-thickness tears as well, or a combination of the two types of tears (e.g. full-thickness tear with a partial-tear aspect or component).

As an initial step, one or more suture implant structures (each having one or more suture limbs) are delivered to a corresponding implant location in a bone area beneath the torn tendon (step 1010). Any method and/or apparatus may be utilized for delivering the suture implant structures according to step 1010, including, without limitation, any of the novel methods and/or cannula and punch devices described above. For example, a screw-type implant may be delivered using the method (700) of FIG. 7 and the cannula-punch system (600) of FIG. 6. In another embodiment, a toggle-type implant may be delivered in a conventional manner using a cannula having a tooth-type extension (see FIG. 4) and a punch having an eccentrically oriented punch tip (see FIG. 5C). In other embodiments, the implant structures may be delivered using known methods and known devices.

Once delivered (step 1010), the implant structure(s) may be properly anchored in place (step 1030) according to the particular type of implant. For instance, screw-type implants may be screwed and tightened and toggle-type implants may be anchored by engaging an underside of the bony implant location.

In the context of a PASTA lesion repair, the implant structure(s) may be advanced through the tendon to their respective implant locations using any method and/or apparatus utilized for delivering the suture implant structures according to step 1010, including, without limitation, any of the novel methods and/or cannula and punch devices described above. As a result, suture limbs emanating from the implant structure(s) will have also been passed through the tendon. For full thickness tears, however, the implant structure(s) may be anchored directly beneath the tendon, without having to be advanced through the tendon. As a result, suture limbs emanating from the implant(s) will not have been passed through the tendon. In such scenarios, an added process step of passing the suture limbs back through the tendon will be required prior to advancing to the step below. However, alternatively, for full-thickness tears, implant structures may be advanced directly through the tendon as well, using any method and/or apparatus utilized for delivering the suture implant structures according to step 1010, including, without limitation, any of the novel methods and/or cannula and punch devices described above.

Next, the suture limbs (e.g., cord-type or band-type) emanating from the implant structures (and through the tendon) are each fastened to limb-anchoring structures (step 1050) and are laterally spanned over the tendon (step 1070). Any type of limb-anchoring structure, including suture-locking and/or suture-interference structures, may be utilized for securing the suture limbs. Once spanned, the sutures are pulled and tensioned (step 1090), via the limb-anchoring structures, and the limb-anchoring structures are affixed to locations that are lateral and/or distal to the implant locations (step 1100). In this manner, the suture limbs are able to secure and compress the tendon to its bony attachment site. Further, since limb-anchoring structures have been utilized, there is no need for knotting the sutures, thereby reducing the overall tendon repair time.

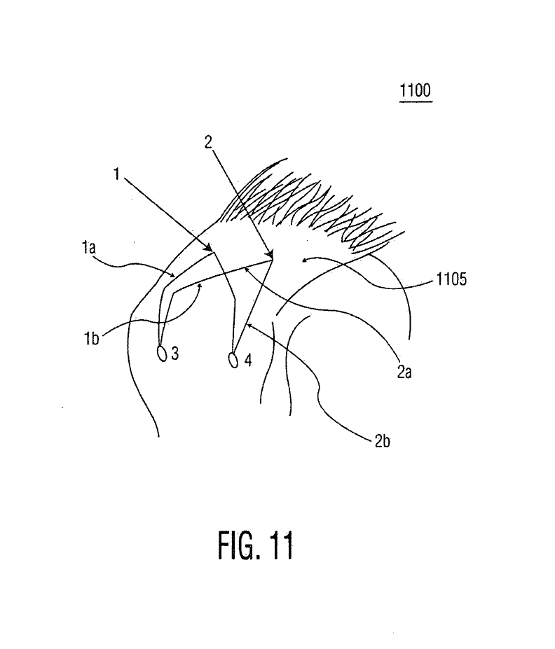

Turning now to FIG. 11, a diagram (1100) illustrating an exemplary PASTA lesion repair conducted using the knotless repair method (1000) discussed above is shown. Underneath the repaired tendon (1105), at locations 1 and 2, two implant structures have been anchored into bone beneath the tendon (1105). For purposes of this illustration, it is assumed that the tendon (1105) had previously been tom, and as a result, location 2 has been located at an anteromedial aspect of the tendon tear, and location 1 has been located at a posteromedial aspect of the tendon tear.

Emanating from each of the two implant locations (1, 2) are two pairs of suture limbs (1a, 1b and 2a, 2b), one each pertaining to each of the two implants. One suture limb from each of the two implant locations is fastened to one of two limb-anchoring structures (3, 4). That is, one suture limb from implant location 1 (1a) and one suture limb from implant location 2 (2a) is each fastened to anchoring structure 3, and a different suture limb from each of implant locations 1 and 2 (1b and 2b, respectively) is fastened to anchoring structure 4. Once the suture limbs (1a, 2a and 1b, 2b) are fastened to their respective limb-anchoring structures (3,4), the suture limbs are spanned over the tendon, pulled and tensioned, via the limb-anchoring structures (3,4), across the tendon. Next, the limb-anchoring structures (3,4) are affixed to their respective locations, which is shown in the diagram (1100), are lateral and/or distal to the implant locations (1, 2). Notably, the number of suture limbs emanating from each location (1,2) may exceed two, and therefore the possible number of suture spanning configurations is potentially more than that described above, particularly when multiple limb-anchoring structures (suture limb-locking or -interference structures) are utilized.

Alternatively, in a variation of the exemplary repair (1100) discussed above, the suture limbs (1a, 1b and 2a, 2b) from each of the implant locations (1, 2) may be fastened to a corresponding one suture limb-locking or -interference structure (or equivalent). In other words, both suture limbs (1a, 1b) emanating from the implant beneath location 1 may be fastened to limb-anchoring structure 4, and the suture limbs (2a, 2b) from the implant beneath location 2 may be fastened to limb-anchoring structure 3.

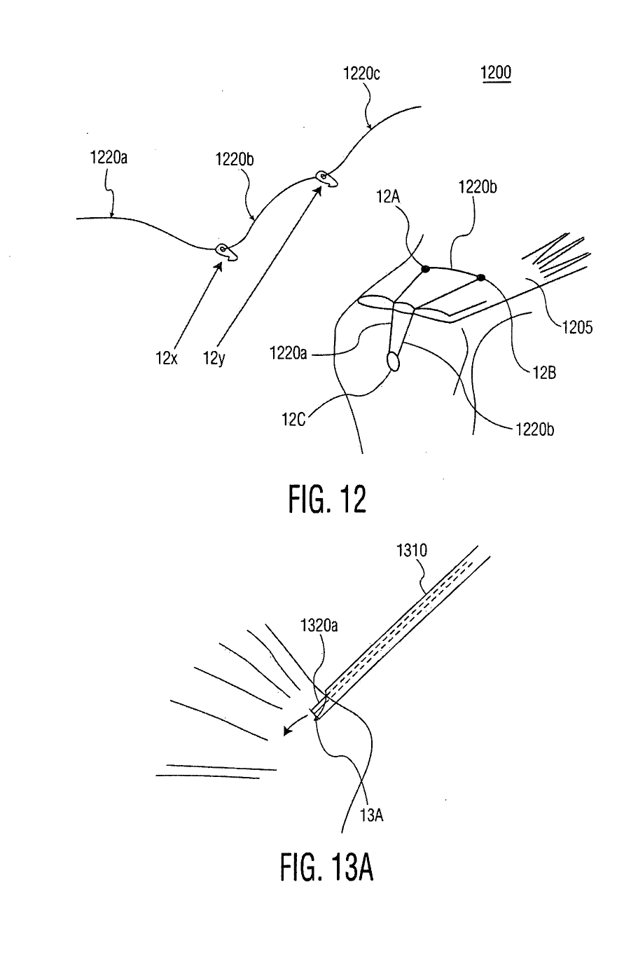

In yet another embodiment, rather than using multiple suture limb-anchoring structures to secure the suture limbs across a tendon (as the exemplary tendon repair of FIG. 11), the suture limbs may be anchored using a single, suture limb-anchoring structure, as shown in FIG. 12. Turning now to FIG. 12, a diagram (1200) showing an exemplary PASTA repair that utilizes two toggle-type implants (12X, 12Y) sequentially loaded and a single suture limb-anchoring structure (12C) is shown. It should be understood, however, that the present diagram (1200) is for illustrational purposes only. More implants and/or suture anchors may be utilized in accordance with the present embodiment dependent on the particular repair. In addition, more suture per implant (12X, 12Y) (e.g., suture anchor, suture limb-locking or -interference structure) may be utilized leaving additional suture (e.g. cord-type or band-type) for tendon spanning.

Returning now to FIG. 12, an exemplary PASTA lesion repair or full thickness tendon repair, or repair of a combination of lesions/tears (e.g., full-thickness tear with partial-tear aspect or component), configured to secure and compress the tendon (1205) in a triangulated manner is shown. This triangulated compression may be accomplished using the various instruments and techniques discussed above. In one embodiment, two toggle-type implants having a single suture threaded between them may be double-loaded into a cannula device. This single suture may be described as having two suture limbs (1220a, 1220c) and a central portion (1220b) that connects the two implants. Using a cannula device, for example, a first of the implants may be advanced through the tendon (1205) and anchored to implant location 12A. Next, the second of the two implants is advanced through the tendon (1205) and anchored to implant location 12B such that the portion of suture (1220b) connecting the two implants rests above the tendon (1205). As noted above, any of the novel instruments and/or delivery methods described above may be utilized in delivering the two implants. In addition, more suture per implant (e.g., suture anchor, suture limb-locking or -interference structure) may be utilized leaving additional suture (e.g. cord-type or band-type) for tendon spanning. The repair is not limited to toggle-type implants, however; suture anchor implant structures and suture limb-locking and -interference structures may be utilized as well, without limitation.

Once the two implants are in place, the suture limbs (1220a, 1220c) are tensioned and fastened to a single limb-anchoring structure. The limb-anchoring structure is then affixed to location 12C, which is lateral and/or distal from implant locations 12A and 12B. In this manner, the suture (1220a-1220c) is able to cinch, compress, and secure the tendon (1205) to the bone area in a triangulated manner.

Alternatively, in a variation of the exemplary repair illustrated in FIG. 12, two implants, each having at least a pair of suture limbs, may be individually advanced and anchored to implant locations 12A and 12B, respectively. Once the implants have been delivered, one suture limb emanating from implant location 12A and one suture limb emanating from implant location 12B may be tied together to form a connecting suture portion (1220b). A second suture limb from implant location 12A (1220a) and a second suture limb from implant location 12B (1220c) may be tensioned and fastened to a single limb-anchoring structure. The limb-anchoring structure may then be affixed to location 12C, which is lateral and/or distal from implant locations 12A and 12B. In this manner, the suture limbs (1220a-1220c) are able to cinch, compress, and secure the tendon (1205) to the bone area in a triangulated manner.

Alternatively, in another variation of the exemplary repair illustrated in FIG. 12, two implants, each having at least a pair of suture limbs, may be individually advanced and anchored to implant locations 12A and 12B, respectively. Once the implants have been delivered, one suture limb emanating from implant location 12A and one suture limb emanating from implant location 12B may be tied together to form a connecting suture portion (1220b). A second suture limb from implant location 12A (1220a) and a second suture limb from implant location 12B (1220c) may be tensioned and tied together to form a second connecting suture portion similar to 1220b. This method is particularly suited for PASTA lesion repair, where the medial portion of the tendon requires tendon compression. This exemplary repair may also be performed utilizing implants having more than two suture limbs associated with it; additional suture limbs may be utilized to repair varying tear patterns, including, but not limited to, full-thickness aspects of a given soft tissue defect.

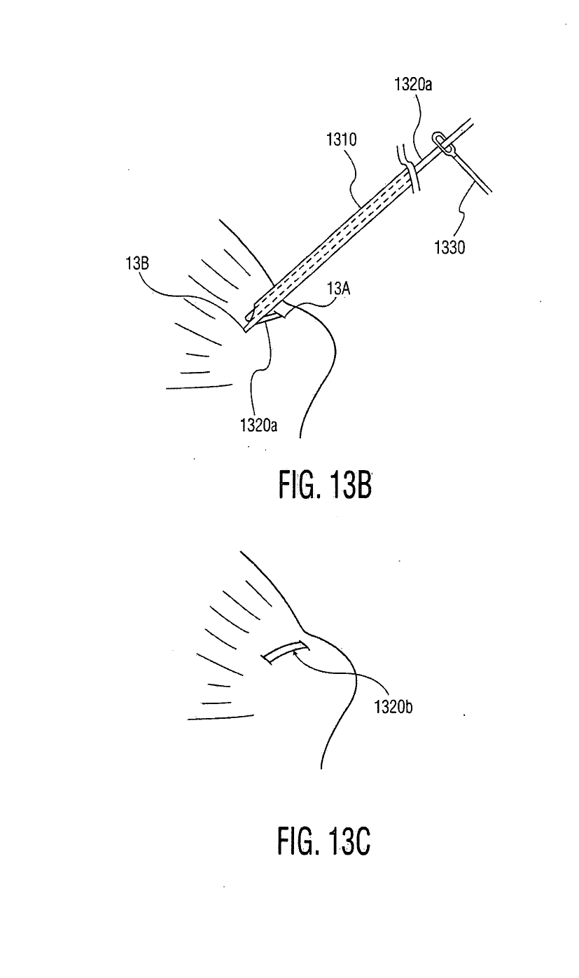

Referring now to FIGS. 13A-13C, another variation of the exemplary repair illustrated in FIG. 12 is shown. One implant (including, but not limited to, suture limb-locking or -interference structures) having at least one pair of suture limbs (1320a) associated with it, may be advanced and anchored to a first implant location 13A (FIG. 13A). The two suture limbs (1320a) from this implant-may then be passed through a suture-limb locking or suture-limb interference structure (1330) (FIG. 13B), which can be placed in a second location 13B, thus creating an anterior-to-posterior suture segment (1320b) in a knotless fashion (FIG. 13C). This method is particularly suited for PASTA lesion repair, where the medial portion of the tendon requires tendon compression. This exemplary repair may also be performed utilizing implants or structures having more than two suture limbs associated with it; additional suture limbs may be utilized to repair varying tear patterns, including, but not limited to, full-thickness aspects of a given soft tissue defect.

As alluded to above, any of the novel instruments and/or techniques described herein may be utilized (alone or in combination) to vastly improve existing tendon repair methods. Indeed, delivering implant devices using, for example, a cannula and punch apparatus of the present invention vastly improves the precision of such deliveries, while at the same time minimizing the risk of additional trauma to the tendon. Further, by spanning suture limbs across a damaged tendon, and then subsequently securing the suture limbs to lateral and/or distal locations using knotless limb-anchoring structures, superior tendon compression and fixation (against bone) is achieved. As a result, the quality of the overall repair and the time required to perform the repair procedure are both greatly improved. Furthermore, since the repair methods of the present invention reduce added tendon trauma and improve tendon-to-bone contact, the tendon itself will have a better opportunity to heal properly and in less time.

Although the invention has been described and illustrated with respect to the exemplary embodiments thereof, it should be understood by those skilled in the art that the foregoing and various other changes, omissions and additions may be made therein and thereto, without parting the spirit and scope of the present invention.

What is claimed is:

1. An implant structure delivery system comprising:
a cannula comprising a hollow, elongated body having a first end and a second end, and being sized and adapted to provide access to a targeted location, said first end being eccentrically tapered and defining a targeting extension, the targeting extension having an extension tip that is distal from the cannula body and adapted for temporarily affixing the cannula to the targeted location.

2. The implant structure delivery system of claim 1, wherein the targeting extension is further adapted for direct insertion through soft tissue or tendon, thereby providing access to the targeted location through an opening in the soft tissue or tendon.

3. The implant structure delivery system of claim 2, wherein the targeting extension is configured with a pointed extension tip adapted for piercing and creating the opening in the soft tissue or tendon.

4. The implant structure delivery system of claim 1, further comprising a cannula handle attached to the second end, said cannula handle being asymmetrically molded for tactile orientation with respect to targeting extension.

5. The implant structure delivery system of claim 1, wherein the cannula body is sized and adapted to permit one or more suture anchor implants to pass therethrough to the targeted location.

6. The implant structure delivery system of claim 5, wherein the suture anchor implants are at least one of a screw-type, plug-type, barb-type, expandable-type, toggle-type, suture locking-type, and suture interference-type implant structure.

7. The implant structure delivery system of claim 2, further comprising:
a punch adapted for creating a pilot hole in the targeted location for receiving a suture anchor implant structure, said punch having a punch tip and being sized and adapted to advance through the cannula body and through the opening in the soft tissue or tendon to the targeted location, the punch tip being adapted to penetrate the targeted location and create a pilot hole.

8. The implant structure delivery system of claim 7, wherein the suture anchor implant is a screw-type implant structure, the system further comprising a tap, said tap being sized and adapted for advancing through the cannula body to the targeted location and for threading the pilot hole in the targeted location created by the punch tip.

9. The implant structure delivery system of claim 7, wherein the punch has an end opposed to said punch tip, said, opposed end being adapted for forcible advancement from a separate instrument.

10. The implant structure delivery system of claim 9, wherein the punch tip is eccentrically tapered.

11. The implant structure delivery system of claim 9, wherein at least one of the cannula and punch comprises one of a circular, elliptical, triangular, rectangular, and rhomboidal cross-sectional profile.

12. The implant structure delivery system of claim 11, wherein the targeted location is a bone area beneath a partially or fully torn tendon.

13. The implant structure delivery system of claim 12, wherein the tendon is one of a rotator cuff tendon and an Achilles tendon.

14. A method of delivering an implant structure to a targeted location, the method comprising:
targeting an implant location;
providing a cannula, said cannula comprising a hollow, elongated body having a first end and a second end, and being sized and adapted to provide access to the targeted implant location, said first end being eccentrically tapered and defining a targeting extension, the targeting extension having an extension tip that is distal from the cannula body and adapted for temporarily affixing the cannula to the targeted location;
localizing the targeted location and temporarily affixing the targeting extension to the targeted location;
providing a punch, said punch having a punch tip and being sized and adapted to advance through the cannula body, said punch tip being adapted for penetrating the targeted location and for creating a pilot hole therein;
advancing the punch through the cannula to the targeted location to create the pilot hole;
removing the punch from the cannula; and
delivering an implant structure through the cannula into the pilot hole created via the punch.

15. The method of claim 14, wherein the targeted location is a bone area beneath a soft tissue or tendon, the method further comprising:
targeting the implant location by piercing and creating an opening, in the soft tissue or tendon; and
localizing the targeted location by inserting the extension tip through the opening created in the soft tissue or tendon.

16. The method of claim 15, wherein the targeting extension is configured with a pointed extension tip adapted for piercing and creating the opening in the soft tissue or tendon, and wherein the targeting and localizing steps occur simultaneously using the pointed extension tip.

17. The method of claim 14, wherein the cannula further comprises a cannula handle attached to the second end, said cannula handle being asymmetrically molded for tactile orientation with respect to targeting extension.

18. The method of claim 14, wherein the implant structure is one or more suture anchor implants selected from the group consisting of a screw-type implant, a plug-type implant, a barb-type implant, an expandable-type implant, a toggle-type implant, suture locking-type implant, and suture interference-type implant structure.

19. The method of claim 18, wherein the suture anchor implant is a screw-type implant structure, the method further comprising:
providing a tap, said tap being sized and adapted for advancing through the cannula body; and
threading the pilot hole created by the punch tip to receive the screw-type implant structure.

20. The method of claim 14, wherein the punch has an end opposed to said punch tip, said opposed end being adapted for forcible advancement, the method further comprising forcibly advancing the punch through the cannula and into the targeted location using said separate instrument.

21. The method of claim 20, wherein the punch tip is tapered along substantially the same angle as that of the targeting extension.

22. The method of claim 21, wherein at least one of the cannula and punch comprises one of a circular, elliptical, triangular, rectangular, and rhomboidal cross-sectional profile.

23. The method of claim 15, wherein the targeted location is a bone area beneath a partially or fully torn tendon.

24. The method of claim 23, wherein the tendon is one of a rotator cuff tendon and an Achilles tendon.

25. A method of repairing a torn tendon, the method comprising:
delivering one or more suture implant structures to one or more targeted implant locations in a bone area beneath a torn tendon, said implant structures each comprising one or more suture limbs;
anchoring the implant structures to the targeted-areas;
laterally spanning the suture limbs over the tendon;
providing one or more suture limb-anchoring structures;
engaging each suture limb to one of the limb-anchoring structures;
pulling and tensioning the suture limbs; and
introducing and affixing the one or more limb-anchoring structures to locations that are at least one of lateral and distal to the targeted locations, thereby securing and compressing the tendon to its bony attachment site.

26. The method of claim 25, wherein two or more implant structures, each having two or more suture limbs are each delivered to respective targeted areas, said targeted areas being at an anteromedial aspect of the tendon tear and at a posteromedial aspect of the tendon tear, the method further comprising:
fastening at least one suture limb from each implant structure to each limb-anchoring structure; and
tensioning the suture limbs and affixing the limb-anchoring structures to at least one of lateral and distal locations.

27. The method of claim 25, wherein two or more implant structures, each having two or more suture limbs, are delivered to respective targeted areas, the targeted areas being at an anteromedial aspect of the tendon tear and at a posteromedial aspect of the tendon tear, the method further comprising:
fastening the two or more suture limbs from each of the implant structures to a single anchoring structure; and
tensioning the suture limbs and affixing the limb-anchoring structure to at least one of a lateral and distal location, thereby compressing and securing the tendon to the bone area.

28. The method of claim 27, wherein one or more suture limbs-from each implant structure remains unfastened to the anchoring structure, the method further comprising tensioning and tying said unfastened suture limbs together, anterior-to-posterior, thereby compressing and securing the tendon to the bone area in a triangulated manner.

29. The method of claim 28, wherein the two or more implant structures comprise at least one of a toggle-type, plug-type, barb-type, expandable-type, screw-type, suture locking-type, and suture interference-type implant structure.

30. The method of claim 25, wherein the delivering step further comprises:
targeting the one or more implant locations,
providing a cannula, said cannula comprising a hollow, elongated body having a first end and a second end, and being sized and adapted to provide access to the targeted implant location, said first end being eccentrically tapered and defining a targeting extension, the targeting extension having an extension tip that is distal from the cannula body and adapted for temporarily affixing the cannula to the targeted location;
for each targeted location:
localizing the targeted location and temporarily affixing the targeting extension in the targeted location;
providing a punch, said punch having a punch tip and being sized and adapted to advance through the cannula body, said punch tip being adapted for penetrating the targeted location and for creating a pilot hole therein;
advancing the punch through the cannula to the targeted location to create the pilot hole;
removing the punch from the cannula; and
delivering the implant structures through the cannula into the pilot hole created via the punch.

31. The method, of claim 30, wherein the implant structure comprises two implants having a single suture threaded between them, said implant structure having a total of two suture limbs, and a portion of the suture connecting the two implants, the method further comprising:
double-loading the two implants into the cannula;
delivering a first of the implants to a first of two targeted locations,
delivering a second of the implants to a second targeted location such that the portion of the suture connecting the two implants rests above the tendon;
tensioning and fastening the two suture limbs to a single limb-anchoring structure, thereby tightening the suture limbs and the portion of the suture threaded between the two implants; and
affixing the limb-anchoring structure to a laterally distal location, thereby cinching, compressing, and securing the tendon to the bone area in a triangulated manner.

32. The method of claim 25, wherein the implant structures comprise one or more suture anchor implants selected: from the group consisting of screw-type, plug-type, barb-type, expandable-type, toggle-type, suture locking-type, and suture interference-type implant structures.

33. The method of claim.,26, wherein at least one of the suture anchor implants is a screw-type implant structure, the method further comprising:
providing a tap, said tap being sized and adapted for advancing through the cannula body; and
threading the pilot hole created by the punch tip to receive the screw-type implant structure.

34. The method of claim 30, wherein the targeting extension is configured with a pointed extension tip adapted for piercing and creating an opening in the soft tissue or tendon, and wherein the targeting and localizing steps occur simultaneously using the pointed extension tip.

35. The method of claim 30, wherein the cannula further comprises a cannula handle attached to the second end, said cannula handle being asymmetrically molded for tactile orientation with respect to targeting extension.

36. The method of claim 30, wherein the punch has an end opposed to said punch tip, said opposed end being adapted for forcible advancement.

37. The method of claim 36, wherein the punch tip is tapered along substantially the same angle as that of the targeting extension.

38. The method of claim 37, wherein at least one of the cannula and punch comprises one of a circular, elliptical, triangular, rectangular, and rhomboidal cross-sectional profile.

39. The method of claim 30, wherein the targeted location is a bone area beneath a partially or fully torn tendon.

40. The method of claim 39, wherein the tendon is one of a rotator cuff tendon and Achilles tendon.

41. The method of claim 25, wherein the suture limb-anchoring structures are one or more of suture-locking and suture-interference structures.

42. The implant structure delivery system of claim 7, wherein the punch tip further comprises a pointed extension for piercing and creating the opening in the soft tissue or tendon.

43. The implant structure delivery system of claim 42, wherein the pointed extension of the punch tip comprises one of a removable pin and a pin-type instrument.

44. The method of claim 14, wherein the punch tip :further comprises a pointed extension adapted for piercing and creating an opening in soft tissue or tendon, and wherein the targeting and localizing steps occur simultaneously using, the punch tip pointed extension.

45. The method of claim 44, wherein the punch tip pointed extension comprises a removable pin, or pin-type instrument, the method further comprising:
inserting the removable pin into the punch tip;
targeting and localizing the targeted location using the punch tip removable pin; and
removing the removable pin from the punch tip prior to creating the pilot hole.

46. The method of claim 44, further comprising:
advancing the punch through the cannula prior to the targeting step, thereby creating a cannula-punch sub-assembly; and
targeting and localizing the targeted location by advancing the cannula-punch sub-assembly through soft tissue or tendon and into said targeted location.

47. The method of claim 30, wherein the punch tip further comprises a pointed extension adapted for piercing and creating an opening in soft tissue or tendon, and wherein the targeting and localizing steps occur simultaneously using the punch tip pointed extension.

48. The method of claim 47, wherein the punch tip pointed extension comprises a removable pin, the method further comprising:
inserting the removable pin into the punch tip;
targeting and localizing the targeted location using the punch tip removable pin; and
removing the removable pin from the punch tip prior to creating the pilot hole.

49. The method of claim 47, further comprising:
advancing the punch through the cannula prior to the targeting step, thereby creating a cannula-punch sub-assembly; and
targeting and localizing the targeted location by advancing the cannula-punch sub-assembly through soft tissue or tendon.

50. The method of claim 39, wherein the tendon is a fully torn tendon, the method further comprising:
passing the suture limbs through the torn tendon prior to pulling and-tensioning said suture limbs.

51. The implant structure delivery system of claim 9, wherein the punch tip is located along a central axis of the punch.

52. The method of claim 25, further comprising:
delivering and anchoring a first implant structure to a first targeted location, said first implant structure having two or more suture limbs;
passing the two or more suture limbs through at least one of a suture-limb locking and suture-limb interference structure;
spanning the two or more suture limbs, anterior-to-posterior, across the torn tendon;
advancing the at least one of a suture-limb locking and suture-limb interference structure through the torn tendon to a second targeted location; and
anchoring said at least one suture-limb locking and suture-limb interference structure at said second targeted location.

53. The method of claim 25, wherein two or more implant structures, each having two or more suture limbs, are delivered to respective targeted areas, the method further comprising tensioning and tying said one suture limb from each implant structure to a corresponding one suture limb from one other of said two or more implant structures, thereby compressing and securing the tendon to the bone area.

54. The method of claim 39, further comprising advancing the cannula, punch, and at least one implant structure to the targeted location through at least a portion of the torn tendon.

55. The implant structure delivery system of claim 3, wherein at least one of the targeting extension and extension tip comprise an independent component that is removably attachable to the cannula.

56. The implant structure delivery system of claim 3, wherein the extension tip is configured as at least one of a tapered tip, beveled tip, a tooth-type tip, and a needle-like tip.

57. The implant structure delivery system of claim 7, wherein the punch tip is configured as at least one of a beveled tip, a needle-like tip, and a thin and flat knife-like tip.

58. The method of claim 25, wherein the one or more of the suture limbs are configured as band-type or cord-type suture limbs.

59. The implant-structure delivery system of claim 9, wherein the punch further comprises a punch handle at said opposed end.

60. The implant structure delivery system of claim 9, wherein the punch further comprises a ridge at said opposed end for advancing the cannula as the punch is advanced.

61. The method of claim 15, further comprising:
providing an obturator for palpating an area on the soft tissue or tendon;
palpating said area on the soft tissue or tendon prior to at least one of the targeting and localizing steps.
